(19) United States
(12) Patent Application Publication
(10) Pub. No.: US 2013/0123942 A1
(43) Pub. Date: May 16, 2013

(54) PROSTHESIS FOOT

(71) Applicants: Chien-Wen CHEN, New Taipei City (TW); Chien-Cheng CHEN, New Taipei City (TW)

(72) Inventors: Chien-Wen CHEN, New Taipei City (TW); Chien-Cheng CHEN, New Taipei City (TW)

(21) Appl. No.: 13/611,152

(22) Filed: Sep. 12, 2012

(30) Foreign Application Priority Data
Nov. 10, 2011 (TW) 100221197

(51) Int. Cl. A61F 2/66

(52) U.S. Cl. 623/53

(57) ABSTRACT

A prosthesis foot includes: a connecting unit including a flexible first plate and a leg-connecting member that is connected to the first plate; a buffer unit including a flexible second plate that has a front portion connected to and disposed under a front portion of the first plate, and an elastic wedge member that is clamped between the first and second plates to form an acute angle between the first and second plates; and a support unit including a flexible third plate that has a rear portion connected to and disposed under a rear portion of the second plate, and an elastic support member that is interposed between the second and third plates to form an acute angle between the second and third plates.

### CROSS-REFERENCE TO RELATED APPLICATION

This application claims priority of Taiwanese Application No. 100221197, filed on Nov. 10, 2011.

### BACKGROUND OF THE INVENTION

1. Field of the Invention

The invention relates to a prosthesis foot, more particularly to a prosthesis foot suitable for use on uneven ground.

2. Description of the Related Art

A conventional prosthesis foot disclosed in Taiwanese Utility Model No. M409061 includes a pair of flexible upper and lower foot plate units. A front portion of the upper foot plate unit is coupled directly to and disposed over a front portion of the lower foot plate unit. The conventional prosthesis foot further includes a buffer member that is clamped between the two foot plate units and that is disposed behind the front portions of the upper and lower foot plate units. With the arrangement of the buffer member, the conventional prosthesis foot is suited for use on uneven ground. However, a toe portion of the prosthesis foot, which corresponds to the front portions of the upper and lower foot plate units and which is not provided with the buffer member, has a poor shock-absorbing ability and may cause discomfort to a user during use.

### SUMMARY OF THE INVENTION

Therefore, the object of the present invention is to provide a prosthesis foot that is suitable for use on uneven ground.

Accordingly, a prosthesis foot of the present invention comprises:

a connecting unit including
a flexible first plate that has a first front portion and a first rear portion opposite to the first front portion, and
a leg-connecting member that is connected to the first rear portion of the first plate;

a buffer unit including
a flexible second plate that has a second front portion connected to and disposed under the first front portion of the first plate, and a second rear portion opposite to the second front portion, and
a first elastic wedge member that is clamped between the first and second plates to form an acute angle between the first and second plates; and

a support unit including
a flexible third plate that has a third rear portion connected to and disposed under the second rear portion of the second plate, and
an elastic support member that is interposed between the second and third plates to form an acute angle between the second and third plates.

### BRIEF DESCRIPTION OF THE DRAWINGS

Other features and advantages of the present invention will become apparent in the following detailed description of the preferred embodiment with reference to the accompanying drawings, of which:

FIG. 1 is an exploded perspective view of a preferred embodiment of a prosthesis foot according to the invention;

FIG. 2 is an assembled perspective view of the preferred embodiment; and

FIG. 3 is a side view of the preferred embodiment.

### DETAILED DESCRIPTION OF THE PREFERRED EMBODIMENT

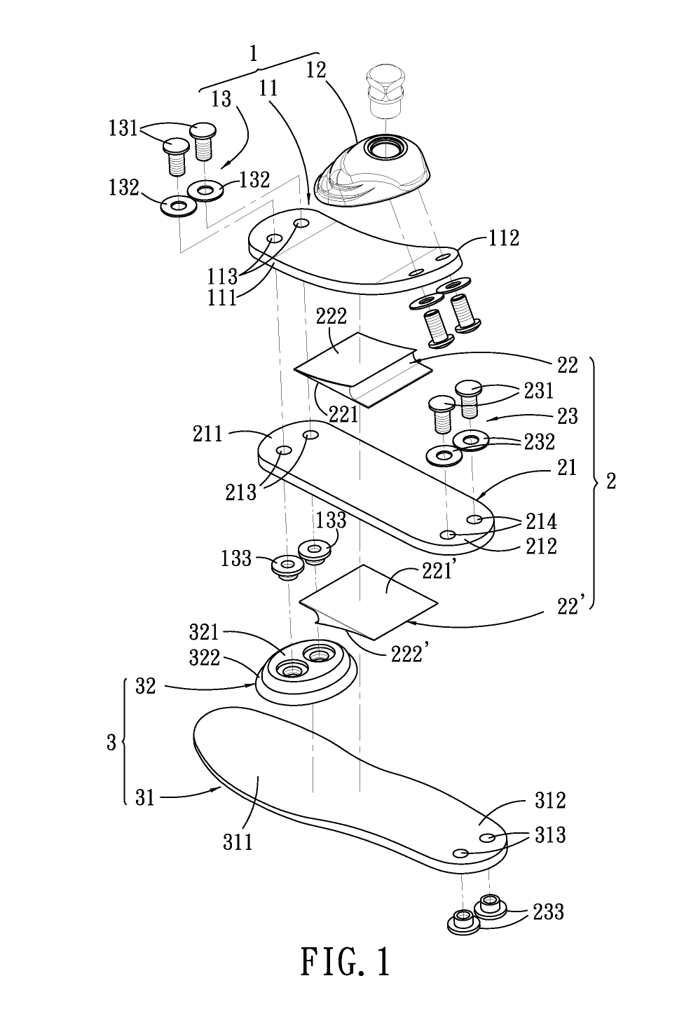

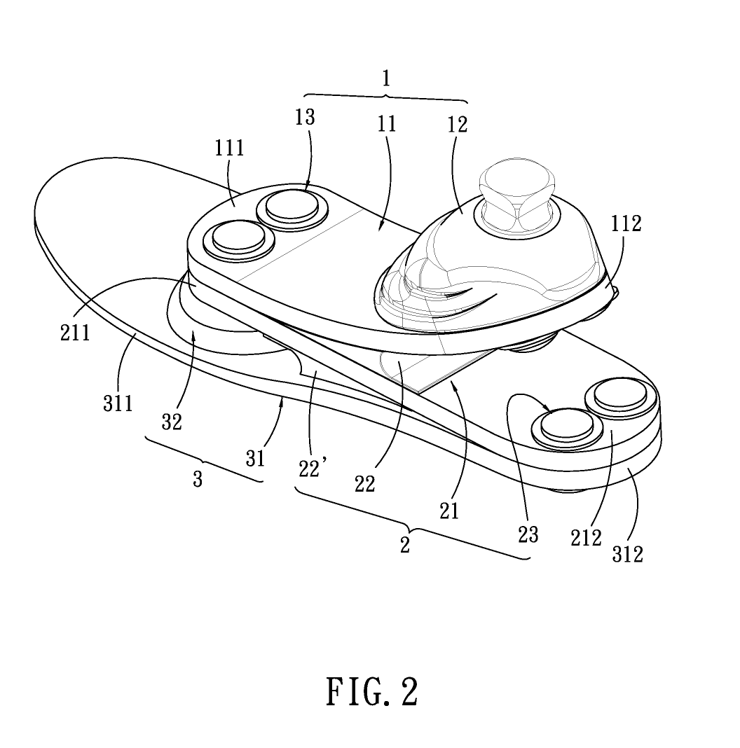

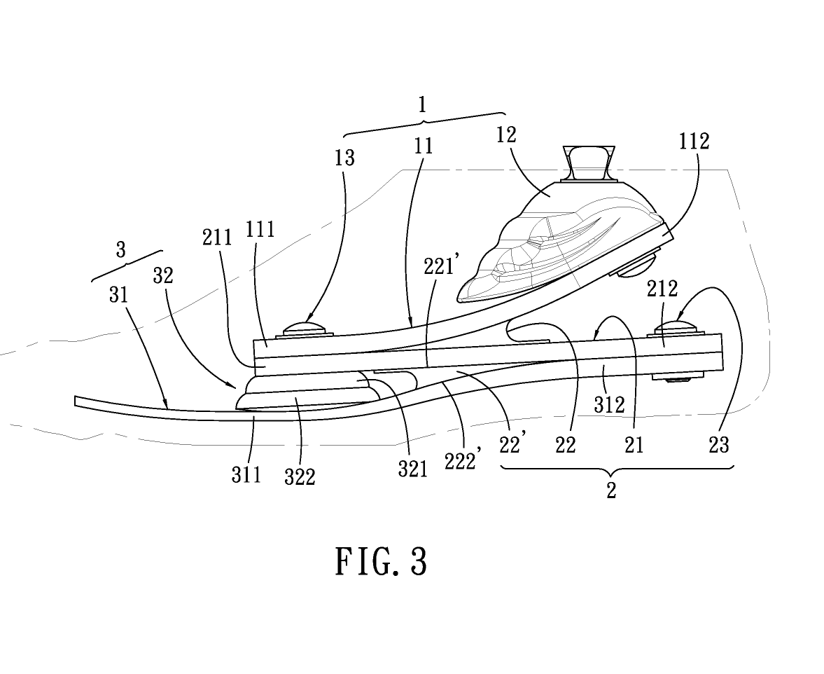

As shown in FIGS. 1, 2 and 3, a preferred embodiment of a prosthesis foot according to the present invention comprises a connecting unit 1, a buffer unit 2, and a support unit 3.

The connecting unit 1 includes a flexible first plate 11 that has a first front portion 111 and a first rear portion 112 opposite to the first front portion 111, and a leg-connecting member 12 that is connected to the first rear portion 112 of the first plate 11 for connecting to a lower limb of a physically disabled person (not shown). The first front portion 111 of the first plate 11 is formed with two first front through holes 113 arranged in a left-right direction.

The buffer unit 2 includes a flexible second plate 21 that has a second front portion 211 disposed under the first front portion 111 of the first plate 11 and a second rear portion 212 opposite to the second front portion 211, and first and second elastic wedge members 22, 22′. The first elastic wedge member 22 is clamped between the first and second plates 11, 21 to form an acute angle between the first and second plates 11, 21. The second front portion 211 of the second plate 21 is formed with two second front through holes 213 arranged in the left-right direction. The second rear portion 212 of the second plate 21 is formed with two second rear through holes 214 arranged in the left-right direction. In this embodiment, each of the first and second plates 11, 21 has a periphery that has front and rear curved segments, and left and right straight segments between the front and rear curved segments.

The support unit 3 includes a flexible third plate 31 that has a third rear portion 312 disposed under the second rear portion 212 of the second plate 21 and a third front portion 311 opposite to the third rear portion 312, and an elastic support member 32 that is interposed between the second and third plates 21, 31 to form an acute angle between the second and third plates 21, 31. The third rear portion 312 of the third plate 31 is formed with two third through holes 313 arranged in the left-right direction.

The support member 32 of the support unit 3 has a first portion 321 that has an oval cross-section, and a second portion 322 that is disposed under the first portion 321 and that has an oval cross-section. The first portion 321 abuts against the second front portion 211 of the second plate 21. The second portion 322 has a bottom surface larger than that of the first portion 321 and abutting against the third front portion 311 of the third plate 31.

The second elastic wedge member 22′ of the buffer unit 2 is clamped between the second and third plates 21, 31 and is disposed behind the support member 32. Each of the first and second elastic wedge members 22, 22′ has a first surface 221, 221′ abutting against the second plate 21, and a second surface 222, 222′ opposite to the first surface 221, 211′, abutting against a respective one of the first and third plates 11, 31, smaller than the first surface 221, 221′, and cooperating with the first surface 221, 221′ to form the acute angles between the first and second plates 11, 21 and between the second and third plates 21, 31.

The connecting unit 1 further includes a first securing unit 13 for securing the first front portion 111 of the first plate 11 to the second front portion 211 of the second plate 21. The first securing unit 13 includes a pair of first fasteners, each of which includes a first bolt 131, a first washer 132, and a first nut 133. For each first fastener, the first bolt 131 extends through the first washer 132, a respective one of the first front through holes 113 and a respective one of the second front through holes 213, and engages threadedly the first nut 133. The first nut 133 is connected to the support member 32.

The buffer unit 2 further includes a second securing unit 23 for securing the second rear portion 212 of the second plate 21 to the third rear portion 312 of the third plate 31. The second securing unit 23 includes a pair of second fasteners, each of which includes a second bolt 231, a second washer 232, and a second nut 233. For each second fastener, the second bolt 231 extends through the second washer 232, a respective one of the second rear through holes 214 and a corresponding one of the third through holes 313, and engages threadedly the second nut 233.

By virtue of the second elastic wedge member 22′ and the support member 32, a toe portion of the prosthesis foot of this invention, which corresponds to the first, second and third front portions 111, 211, 311 of the first, second and third plates 11, 21, 31, has an improved shock-absorbing ability and is suitable for use on uneven ground.

It should be noted that the materials of the first and second elastic wedge members 22, 22′ and the support member 32 may vary depending on requirements. For example, a stiffer material may be chosen for the first and second elastic wedge members 22, 22′ and the support member 32 to suit users of the prosthesis foot of the invention who are relatively fast walkers, while a softer material may be chosen for the first and second elastic wedge members 22, 22′ and the support member 32 to suit users who prefer minimum discomfort during movement with the prosthesis foot of the invention.

It should be further noted that the numbers of the first fasteners for each first securing unit 13 and the second fasteners for each second securing unit 23 may vary in other embodiments of this invention.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A prosthesis foot comprising:
a connecting unit including
a flexible first plate that has a first front portion and a first rear portion opposite to said first front portion, and
a leg-connecting member that is connected to said first rear portion of said first plate;
a buffer unit including
a flexible second plate that has a second front portion connected to and disposed under said first front portion of said first plate, and a second rear portion opposite to said second front portion, and
a first elastic wedge member that is clamped between said first and second plates to form an acute angle between said first and second plates; and
a support unit including
a flexible third plate that has a third rear portion connected to and disposed under said second rear portion of said second plate, and
an elastic support member that is interposed between said second and third plates to form an acute angle between said second and third plates.

2. The prosthesis foot as claimed in claim 1, wherein:
said support member has
a first portion that has an oval cross-section, and
a second portion that is disposed under said first portion and that has an oval cross-section; and
said first portion abuts against said second front portion of said second plate, said second portion having a bottom surface that is larger than that of said first portion and that abuts against said third plate.

3. The prosthesis foot as claimed in claim 1, wherein said first elastic wedge member has a first surface abutting against said second plate, and a second surface opposite to said first surface, smaller than said first surface and cooperating with said first surface to form the acute angle between said first and second plates.

4. The prosthesis foot as claimed in claim 1, wherein said buffer unit further includes a second elastic wedge member clamped between said second and third plates and disposed behind said support member.

5. The prosthesis foot as claimed in claim 4, wherein said connecting unit further includes a first securing unit for securing said first front portion of said first plate to said second front portion of said second plate, said buffer unit further including a second securing unit for securing said second rear portion of said second plate to said third rear portion of said third plate.

6. The prosthesis foot as claimed in claim 5, wherein said first securing unit includes a pair of first fasteners that are arranged in a left-right direction and that are connected to said support member, said second securing unit including a pair of second fasteners that are arranged in the left-right direction.

7. The prosthesis foot as claimed in claim 6, wherein:
said first front portion of said first plate is formed with two first front through holes;
said second front portion of said second plate is formed with two second front through holes, and said second rear portion of said second plate is formed with two second rear through holes;
said third rear portion of said third plate is formed with two third through holes;
each of said first fasteners includes a first bolt, a first washer, and a first nut, said first bolt extending through said first washer, a respective one of said first front through holes and a respective one of said second front through holes, and engaging threadedly said first nut; and
each of said second fasteners includes a second bolt, a second washer, and a second nut, said second bolt extending through said second washer, a respective one of said second rear through holes and a respective one of said third through holes of said third plate, and engaging threadedly said second nut.

8. The prosthesis foot as claimed in claim 1, wherein said connecting unit further includes a first securing unit for securing said first front portion of said first plate to said second front portion of said second plate, said buffer unit further including a second securing unit for securing said second rear portion of said second plate to said third rear portion of said third plate.

9. The prosthesis foot as claimed in claim 8, wherein said first securing unit includes a pair of first fasteners that are arranged in a left-right direction and that are connected to said support member, said second securing unit including a pair of second fasteners that are arranged in the left-right direction.

10. The prosthesis foot as claimed in claim 9, wherein:
said first front portion of said first plate is formed with two first front through holes;
said second front portion of said second plate is formed with two second front through holes, and said second rear portion of said second plate is formed with two second rear through holes;
said third rear portion of said third plate is formed with two third through holes;
each of said first fasteners includes a first bolt, a first washer, and a first nut, said first bolt extending through said first washer, a respective one of said first front through holes, and a respective one of said second front through holes, and engaging threadedly said first nut; and
each of said second fasteners includes a second bolt, a second washer, and a second nut, said second bolt extending through said second washer, a respective one of said second rear through holes, and a respective one of said third through holes of said third plate, and engaging threadedly said second nut.
